(19) United States
(12) Patent Application Publication
(10) Pub. No.: US 2023/0090285 A1
(43) Pub. Date: Mar. 23, 2023

(54) DEHYDROGENATION CATALYST SYSTEMS AND METHODS FOR USING THEM

(71) Applicant: Clariant International Ltd, Muttenz (CH)

(72) Inventors: Rong XING, Louisville, KY (US); Vladimir FRIDMAN, Louisville, KY (US)

(21) Appl. No.: 17/801,845

(22) PCT Filed: Apr. 21, 2021
(86) PCT No.: PCT/EP2021/060346

§ 371 Date: Aug. 24, 2022

Related U.S. Application Data
(60) Provisional application No. 63/018,656, filed on May 1, 2020.

(51) Int. Cl. B01J 23/63; B01J 21/12; B01J 23/72; B01J 23/02; B01J 35/02; B01J 37/08; B01J 37/02; C07C 5/32

(52) U.S. Cl. CPC B01J 23/63; B01J 21/12; B01J 23/72; B01J 23/02; B01J 35/026; B01J 37/088; B01J 37/0201; C07C 5/325; C07C 2523/63

(57) ABSTRACT

The present disclosure relates to mixed-bed systems comprising a particulate dehydrogenation catalyst based on one or more certain group 13 and 14 elements that further include additional metal components and a particulate non-catalytic additive comprising a heat-generating material, and to methods for dehydrogenating hydrocarbons using such systems. One aspect of the disclosure provides a mixed-bed system comprising a particulate dehydrogenation catalyst and a particulate non-catalytic additive. The particulate dehydrogenation catalyst includes a primary species P1 selected from Ga, In, TI, Ge, Sn Pb, and any mixture thereof; a primary species P2 selected from the lanthanides and any mixture thereof; a promoter M1 selected from Ni, Pd, Pt, La, Ir, Zn, Fe, Rh, Ru, Mn, Co, W, and any mixture thereof; and a promoter M2 selected from Li, Na, K, Rb, Cs, Be, Mg, Ca, Sr, Ba, and any mixture thereof on a support S1 selected from silica, alumina, zirconia, titania, yttria, and any mixture thereof. The particulate non-catalytic additive includes a heat-generating material and a carrier selected from inorganic oxides, clays, and any mixture thereof.

### BACKGROUND OF THE DISCLOSURE

### Field of the Disclosure

This disclosure relates generally to catalyst systems and methods for using them. More particularly, the present disclosure relates to mixed-bed systems comprising a particulate dehydrogenation catalyst based on one or more certain group 13 and 14 elements that further include additional metal components and a particulate non-catalytic additive comprising a heat-generating material, and to methods for dehydrogenating hydrocarbons using such systems.

### Technical Background

Alkane dehydrogenation is a recognized process for production of a variety of useful hydrocarbon products, such as in the dehydrogenation of propane to make propene for use in the polymer industry, dehydrogenation of n-butane to produce n-butene or alkylate and butadiene useful in tire production, and the dehydrogenation of isobutane to make isobutylene suitable for conversion to methyl tert-butyl ether, isooctane, and alkylates to supplement and enrich gasolines. Current commercial catalysts useful for catalytic dehydrogenation of light alkanes include CrOx/Al₂O₃ and Pt—Sn/Al₂O₃ catalysts, which have been in use for decades.

CrOx/Al₂O₃ dehydrogenation catalysts typically contain a majority of their chromium in the Cr(III) oxidation state on the alumina surface. However, there typically remains a small amount of Cr(VI), which is carcinogenic and thus presents health risks during catalyst handling and operation. They also can cause significant environmental pollution.

Gallium-based dehydrogenation catalysts have been known for two decades. They are generally not hazardous, and their application presents no significant environmental issue. However, these catalysts have limitations in activity and stability, especially for the commercially important dehydrogenation of propane. For example, the reaction temperature necessary to maintain a desired propylene yield for a gallium-based dehydrogenation catalyst often increases with the number of reaction-regeneration cycles to which the catalyst is subjected.

Accordingly, there remains a need for dehydrogenation catalyst systems that provide improved activity and stability, especially in the dehydrogenation of propane.

### SUMMARY OF THE DISCLOSURE

The scope of the present disclosure is not affected to any degree by the statements within the summary.

In one aspect, the disclosure provides a mixed-bed system comprising
a particulate dehydrogenation catalyst comprising Ga, In, TI, Ge, Sn, Pb, or any mixture thereof (e.g., Ga) as an active metal, disposed on a support; and
a particulate non-catalytic additive comprising
   a heat-generating material; and
   a carrier selected from inorganic oxides, clays, and any mixture thereof.

In one aspect, the disclosure provides a mixed-bed system comprising

a particulate dehydrogenation catalyst comprising
a primary species P1 selected from Ga, In, TI, Ge, Sn, Pb, and any mixture thereof, present in the particulate dehydrogenation catalyst in an amount within the range of 0.05 wt. % to 20 wt. %, calculated as elemental metal on a calcined basis;
a primary species P2 selected from the lanthanides and any mixture thereof, present in the particulate dehydrogenation catalyst in an amount within the range of 0.05 wt. % to 10 wt. %, calculated as elemental metal on a calcined basis;
a promoter M1 selected from Ni, Pd, Pt, La, Ir, Zn, Fe, Rh, Ru, Mn, Co, W, and any mixture thereof, present in the particulate dehydrogenation catalyst in an amount within the range of 10 ppm to 500 ppm, calculated as elemental metal on a calcined basis;
a promoter M2 selected from Li, Na, K, Rb, Cs, Be, Mg, Ca, Sr, Ba, and any mixture thereof, present in the particulate dehydrogenation catalyst in an amount within the range of 0.05 wt. % to 3 wt. %, calculated as elemental metal on a calcined basis; and
a support S1 selected from silica, alumina, zirconia, titania, yttria, and any mixture thereof, present in the particulate dehydrogenation catalyst in an amount within the range of 60 wt. % to 99 wt. %, calculated as oxide on a calcined basis; and
   a particulate non-catalytic additive comprising
a heat-generating material; and
a carrier selected from inorganic oxides, clays, and any mixture thereof.

Another aspect of the disclosure is a method for dehydrogenating hydrocarbons, the method comprising contacting a hydrocarbon feed with a mixed-bed system as described herein; and performing a plurality of reaction cycles, each reaction cycle comprising contacting a hydrocarbon feed with the system to dehydrogenate the hydrocarbon feed and to form a deactivated system comprising a reduced heat-generating material and reaction by-products (e.g., coke) adsorbed onto the surface of the mixed-bed system; and contacting the deactivated system with an oxygen-containing gas (e.g., air) to remove adsorbed reaction by-products (e.g., coke) and to oxidize the heat-generating material.

Other aspects of the disclosure will be apparent to the person of ordinary skill in the art in view of the disclosure herein.

### BRIEF DESCRIPTION OF THE DRAWINGS

FIG. 1 is a set of graphs showing the temperature profile of a reaction-regeneration cycle of a mixed-bed system described herein (top graph) and a conventional catalyst (bottom graph), as measured at the top (top line), middle (middle line), and bottom (bottom line) of the catalyst bed.

FIG. 2 is a set of graphs comparing the propylene yield (left) and the propylene selectivity (right) of a mixed-bed system described herein and conventional catalysts.

### DETAILED DESCRIPTION

In various aspects, the disclosure relates to mixed-bed systems that include a particulate dehydrogenation catalyst and a particulate non-catalytic additive. The particulate dehydrogenation catalyst includes a primary species selected from certain group 13 and group 14 elements, disposed on a support. The particulate non-catalytic additive includes a heat-generating material and a carrier. The present inventors have determined that such systems, which may advantageously be free of chromium-containing materials, can exhibit performance comparable to or even better than conventional, commercially available catalysts, while avoiding unnecessary energy expenditure.

Accordingly, one aspect of the disclosure provides a mixed-bed system comprising a particulate dehydrogenation catalyst comprising Ga, In, TI, Ge, Sn, Pb, or any mixture thereof as an active metal, disposed on a support; and a particulate non-catalytic additive comprising a heat-generating material and a carrier selected from inorganic oxides, clays, and any mixture thereof.

One particular aspect of the disclosure provides a mixed-bed system comprising a particulate dehydrogenation catalyst and a particulate non-catalytic additive. The particulate dehydrogenation catalyst includes a primary species, P1, selected from Ga, In, TI, Ge, Sn, Pb, and any mixture thereof, present in the particulate dehydrogenation catalyst in an amount within the range of 0.05 wt. % to 20 wt. %, calculated as elemental metal on a calcined basis. The particulate dehydrogenation catalyst also includes a primary species, P2, selected from the lanthanides (e.g., La, Ce, Nd) and any mixture thereof, present in the particulate dehydrogenation catalyst in an amount within the range of 0.05 wt. % to 10 wt. %, calculated as elemental metal. The particulate dehydrogenation catalyst also includes a promoter, M1, selected from Ni, Pd, Pt, and any mixture thereof, present in the particulate dehydrogenation catalyst in an amount within the range of 1 ppm to 500 ppm, calculated as elemental metal on a calcined basis. The particulate dehydrogenation catalyst also includes a promoter, M2, selected from Li, Na, K, Rb, Cs, Be, Mg, Ca, Sr, Ba, and any mixture thereof, present in the particulate dehydrogenation catalyst in an amount within the range of 0.05 wt. % to 3 wt. %, calculated as elemental metal on a calcined basis. And the particulate dehydrogenation catalyst includes a support, S1, present in the particulate dehydrogenation catalyst in an amount within the range of 60 wt. % to 99 wt. %, calculated as oxide on a calcined basis. The particulate non-catalytic additive comprises one or more components selected form inorganic oxides and clay; and a heat-generating material. The present inventors have determined that the heat-generating material can advantageously help to drive dehydrogenation reactions mediated by the mixed-bed systems described herein at relatively lower temperatures.

As used herein, the terms “alumina” and “silica” include aluminum oxide and silicon oxide, respectively. As used herein, the term “oxide,” including, e.g., “mixed oxide,” “aluminum oxide,” “silicon oxide,” etc., includes oxides in all forms and crystalline phases. For example, “aluminum oxide” includes Al₂O3, Al₂Oₓ wherein x is within the range of 1 to 3, etc. Unless otherwise indicated, regardless of the actual stoichiometry of the oxide, oxides are calculated as the most stable oxide for purposes of weight percent determinations. For example, the person of ordinary skill in the art will appreciate that a non-stoichiometric oxide of aluminum, or even another form of aluminum, may still be calculated as Al₂O₃ for purposes of weight percent determinations. Moreover, unless otherwise indicated, the compositions are described on an as-calcined basis.

Without intending to be bound by theory, the present inventors believe that P1 acts as a primary catalytic species in dehydrogenation reactions mediated by the mixed-bed systems described herein. In certain embodiments as otherwise described herein, P1 is selected from Ga, Ge, In, Sn, TI, and any mixture thereof. For example, in certain embodiments, P1 is (or includes) Ga. In other embodiments, P1 is (or includes) In, Sn, and/or TI. For example, in certain embodiments, P1 is (or includes) Ga and Sn.

In certain embodiments of the systems as otherwise described herein, P1 is present in the particulate dehydrogenation catalyst in an amount within the range of 0.05 wt. % to 17.5 wt. %, or 0.05 wt. % to 15 wt. %, or 0.05 wt. % to 12.5 wt. %, or 0.05 wt. % to 10 wt. %, or 0.05 wt. % to 7.5 wt. %, or 0.05 wt. % to 5 wt. %, or 0.1 wt. % to 20 wt. %, or 0.25 wt. % to 20 wt. %, or 0.5 wt. % to 20 wt. %, or 0.75 wt. % to 20 wt. %, or 1 wt. % to 20 wt. %, or 1.5 wt. % to 20 wt. %, or 2 wt. % to 20 wt. %, or 2.5 wt. % to 20 wt. %, or 5 wt. % to 20 wt. %, or 7.5 wt. % to 20 wt. %, or 10 wt. % to 20 wt. %, or 12.5 wt. % to 20 wt. %, or 15 wt. % to 20 wt. %, or 0.1 wt. % to 17.5 wt. %, or 0.1 wt. % to 15 wt. %, or 0.1 wt. % to 12.5 wt. %, or 0.1 wt. % to 10 wt. %, or 0.5 wt. % to 7.5 wt. %, calculated as elemental metal on a calcined basis.

Without intending to be bound by theory, the present inventors believe that P2 acts as a primary catalytic species in dehydrogenation reactions mediated by the mixed-bed systems described herein. In certain embodiments as otherwise described herein, P2 is selected from La, Ce, Nd, and any mixture thereof. For example, in certain embodiments, P2 is (or includes) Ce. In other embodiments, P2 is (or includes) La. For example, in certain embodiments as otherwise described herein, P2 is (or includes) Ce and La. In other embodiments, P2 is (or includes) Nd.

In certain embodiments as otherwise described herein, P2 is present in the particulate dehydrogenation catalyst in an amount within the range of 0.05 wt. % to 9 wt. %, or 0.05 wt. % to 8 wt. %, or 0.05 wt. % to 7 wt. %, or 0.05 wt. % to 6 wt. %, or 0.05 wt. % to 5 wt. %, or 0.05 wt. % to 4 wt. %, or 0.05 wt. % to 3 wt. %, or 0.05 wt. % to 2 wt. %, or 0.05 wt. % to 1 wt. %, or 0.1 wt. % to 10 wt. %, or 0.25 wt. % to 10 wt. %, or 0.5 wt. % to 10 wt. %, or 0.75 wt. % to 10 wt. %, or 1 wt. % to 10 wt. %, or 1.5 wt. % to 10 wt. %, or 2 wt. % to 10 wt. %, or 3 wt. % to 10 wt. %, or 4 wt. % to 10 wt. %, or 5 wt. % to 10 wt. %, or 0.1 wt. % to 9 wt. %, or 0.1 wt. % to 8 wt. %, or 0.1 wt. % to 7 wt. %, or 0.1 wt. % to 6 wt. %, or 0.25 wt. % to 5 wt. %, calculated as elemental metal on a calcined basis.

In certain embodiments as otherwise described herein, M1 is selected from Pt, Ir, La, Zn, Fe, Rh, Pd, Ru, and any mixture thereof. In certain embodiments as otherwise described herein, M1 is selected from Pd, Pt, Ir, La, and any mixture thereof. In certain embodiments as otherwise described herein, M1 is selected from Pd, Pt, and a mixture thereof. For example, in certain embodiments, M1 is (or includes) Pd. In other embodiments, M1 is (or includes) Pt.

In certain embodiments as otherwise described herein, M1 is present in the particulate dehydrogenation catalyst in an amount within the range of 1 ppm to 450 ppm, or 1 ppm to 400 ppm, or 1 ppm to 350 ppm, or 1 ppm to 300 ppm, or 1 ppm to 250 ppm, or 1 ppm to 200 ppm, or 1 ppm to 150 ppm, or 1 ppm to 100 ppm, or 25 ppm to 500 ppm, or 50 ppm to 500 ppm, or 75 ppm to 500 ppm, or 100 ppm to 500 ppm, or 150 ppm to 500 ppm, or 200 ppm to 500 ppm, or 250 ppm to 500 ppm, or 300 ppm to 500 ppm, or 250 ppm to 500 ppm, or 25 ppm to 450 ppm, or 50 ppm to 400 ppm, or 75 ppm to 350 ppm, or 100 ppm to 300 ppm, calculated as elemental metal on a calcined weight basis.

In certain embodiments as otherwise described herein, M2 is selected from K, Na, Ce, Li, Ca, Mg, Sr, Ba, and any mixture thereof. In certain embodiments as otherwise described herein, M2 is selected from Li, Na, K, Cs, Ba, and any mixture thereof. For example, in certain embodiments, M2 is (or includes) K. In other embodiments, M2 is (or includes) Ba and K (e.g., where P2 is, or includes, Ce).

In certain embodiments as otherwise described herein, M2 is present in the particulate dehydrogenation catalyst in an amount within the range of 0.05 wt. % to 2.25 wt. %, or 0.05 wt. % to 2 wt. %, or 0.05 wt. % to 1.75 wt. %, or 0.05 wt. % to 1.5 wt. %, or 0.05 wt. % to 1.25 wt. %, or 0.05 wt. % to 1 wt. %, or 0.1 wt. % to 2.5 wt. %, or 0.25 wt. % to 2.5 wt. %, or 0.5 wt. % to 2.5 wt. %, or 0.75 wt. % to 2.5 wt. %, or 1 wt. % to 2.5 wt. %, or 1.25 wt. % to 2.5 wt. %, or 1.5 wt. % to 2.5 wt. %, or 1.75 wt. % to 2.5 wt. %, or 2 wt. % to 2.5 wt. %, or 0.1 wt. % to 2 wt. %, or 0.1 wt. % to 1.75 wt. %, or 0.1 wt. % to 1.5 wt. %, or 0.1 wt. % to 1.25 wt. %, or 0.1 wt. % to 1 wt. %, calculated as elemental metal on a calcined basis.

In certain embodiments as otherwise described herein, P1 is selected from Ga, In, TI, Ge, Sn, Pb, and any mixture thereof; P2 is selected from the lanthanides and any mixture thereof; M1 is selected from Ni, Pd, Pt, and any mixture thereof; M2 is selected from Li, Na, K, Rb, Cs, Be, Mg, Ca, Sr, Ba; and S1 is a mixture of silica and alumina.

For example, in certain embodiments as otherwise described herein, P1 (e.g., Ga) is present in the particulate dehydrogenation catalyst in an amount within the range of 0.1 wt. % to 10 wt. %, or 0.5 wt. % to 9 wt. %, or 0.75 wt. % to 8 wt. %, or 1 wt. % to 7 wt. %, or 2.5 wt. % to 5 wt. %, calculated as elemental metal on a calcined basis. In certain such embodiments, P2 (e.g., Ce, or La and Ce) is present in the particulate dehydrogenation catalyst in an amount within the range of 0.1 wt. % to 6 wt. %, or 0.5 wt. % to 5 wt. %, or 1 wt. % to 4 wt. %, or 1 wt. % to 3 wt. %, calculated as elemental metal on a calcined basis. In certain such embodiments, M1 (e.g., Pt) is present in the particulate dehydrogenation catalyst in an amount within the range of 1 ppm to 500 ppm, 25 ppm to 450 ppm, or 50 ppm to 400 ppm, calculated as elemental metal on a calcined weight basis. In certain such embodiments, M2 (e.g., K, or K and Ba) is present in the particulate dehydrogenation catalyst in an amount within the range of 0.05 wt. % to 2.5 wt. %, or 0.25 wt. % to 2.5 wt. %, or 0.1 wt. % to 1.5 wt. %, calculated as elemental metal on a calcined basis.

In certain embodiments as otherwise described herein, P1 is (or includes) Ga, and if P2 includes Ca, the particulate dehydrogenation catalyst further comprises La and/or Ba. For example, in certain such embodiments, P1 is (or includes) Ga, P2 is (or includes) Ce, and the particulate dehydrogenation catalyst further comprises La and/or Ba.

In certain embodiments as otherwise described herein, S1 includes a mixture of silica and alumina. The person of ordinary skill in the art will further appreciate that a “mixture,” e.g., of silica and alumina, includes homogeneous and heterogeneous mixtures. For example, a support S1 including a mixture of silica and alumina may comprise a covalently bound network including both silicon and aluminum atoms (e.g., —Si—O-AI—), and/or discrete domains of both silica and alumina.

In certain embodiments as otherwise described herein, the amount of silica present in S1 is within the range of 1 wt. % to 70 wt. % of S1. For example, in certain embodiments as otherwise described herein, the amount of silica present in S1 is within the range of 1 wt. % to 65 wt. %, or 1 wt. % to 60 wt. %, or 1 wt. % to 55 wt. %, or 1 wt. % to 50 wt. %, or 1 wt. % to 40 wt. %, or 1 wt. % to 30 wt. %, or 1 wt. % to 20 wt. %, or 1 wt. % to 10 wt. %, or 2.5 wt. % to 70 wt. %, or 5 wt. % to 70 wt. %, or 7.5 wt. % to 70 wt. %, or 10 wt. % to 70 wt. %, or 15 wt. % to 70 wt. %, or 20 wt. % to 70 wt. %, or 30 wt. % to 70 wt. %, or 40 wt. % to 70 wt. % of S1, or 50 wt. % to 70 wt. %. In certain embodiments as otherwise described herein, the amount of alumina present in S1 is within the range of 30 wt. % to 99 wt. % of S1. For example, in certain embodiments as otherwise described herein, the amount of alumina present in S1 is within the range of 30 wt. % to 97.5 wt. %, or 30 wt. % to 95 wt. %, or 30 wt. % to 90 wt. %, or 30 wt. % to 85 wt. %, or 30 wt. % to 80 wt. %, or 30 wt. % to 70 wt. %, or 30 wt. % to 60 wt. %, or 40 wt. % to 99 wt. %, or 50 wt. % to 99 wt. %, or 60 wt. % to 99 wt. %, or 70 wt. % to 99 wt. %, or 80 wt. % to 99 wt. %, or 85 wt. % to 99 wt. %, or 90 wt. % to 99 wt. %, or 50 wt. % to 97.5 wt. %, or 60 wt. % to 95 wt. %, or 70 wt. % to 90 wt. %.

In certain embodiments as otherwise described herein, the total amount of alumina and silica in S1 is at least 80 wt. % of S1. For example, in certain as otherwise described herein, the total amount of alumina and silica in S1 is at least 85 wt. %, at least 90 wt. %, at least 92.5 wt. %, at least 95 wt. %, at least 97.5 wt. %, at least 98 wt. %, or at least 99 wt. % of S1.

In certain embodiments as otherwise described herein, S1 is present in the particulate dehydrogenation catalyst in an amount within the range of 50 wt. % to 97.5 wt. %, or 50 wt. % to 95 wt. %, or 50 wt. % to 90 wt. %, or 50 wt. % to 85 wt. %, or 50 wt. % to 80 wt. %, or 50 wt. % to 75 wt. %, or 55 wt. % to 99 wt. %, or 60 wt. % to 99 wt. %, or 65 wt. % to 99 wt. %, or 70 wt. % to 99 wt. %, or 75 wt. % to 99 wt. %, or 80 wt. % to 99 wt. %, or 85 wt. % to 99 wt. %, or 90 wt. % to 99 wt. %.

In certain embodiments as otherwise described herein, S1 includes zirconia. For example, in certain embodiments, the amount of zirconia present in S1 is at least 50 wt. %, or at least 55 wt. %, or at least 60 wt. %, or at least 65 wt. %., or at least 70 wt. %, or at least 75 wt. %, or at least 80 wt. %, or at least 85 wt. %, or at least 90 wt. %, or at least 95 wt. % of S1. In certain embodiments as otherwise described herein, the amount of zirconia present in S1 is within the range of 50 wt. % to 99 wt. %, or 60 wt. % to 99 wt. %, or 70 wt. % to 99 wt. %, or 80 wt. % to 99 wt. %, or 90 wt. % to 99 wt. %, or 50 wt. % to 95 wt. %, or 50 wt. % to 90 wt. %, or 50 wt. % to 85 wt. %, or 50 wt. % to 80 wt. %, or 50 wt. % to 75 wt. % of S1.

In certain embodiments as otherwise described herein, S1 includes titania. For example, in certain embodiments, the amount of titania present in S1 is within the range of 10 wt. % to 75 wt. %, or 25 wt. % to 75 wt. %, or 50 wt. % to 75 wt. %, or 10 wt. % to 65 wt. %, or 10 wt. % to 55 wt. %, or 10 wt. % to 50 wt. %, or 10 wt. % to 40 wt. %, or 15 wt. % to 65 wt. %, or 25 wt. % to 50 wt. % of S1. In certain embodiments as otherwise described herein, S1 includes zirconia, present in an amount within the range of 50 wt. % to 75 wt. %, and titania, present in an amount within the range of 25 wt. % to 50 wt. %.

In certain embodiments as otherwise described herein, S1 includes zirconia, alumina, and silica, present in a total amount of at least 80 wt. %, or at least 85 wt. %, or at least 90 wt. %, or at least 95 wt. %, or at least 97.5 wt. %, or at least 99 wt. % of S1. In other embodiments, the total amount of silica and alumina present in S1 is less than 5 wt. %, or less than 4 wt. %, or less than 2 wt. %, or less than 0.5 wt. %, or less than 0.25 wt. % of S1. For example, in certain embodiments, S1 is substantially free from silica and alumina.

The person of ordinary skill in the art will appreciate that the mixed-bed system may, in some embodiments as otherwise described herein, be substantially free of Cr. Chromium-free systems are especially desirable from an environmental perspective. For example, in certain embodiments of the systems as otherwise described herein, the mixed-bed system includes less than 1 wt. %, or less than 0.9 wt. %, or less than 0.8 wt. %, or less than 0.7 wt. %, or less than 0.6 wt. %, or less than 0.5 wt. %, or less than 0.4 wt. %, or less than 0.3 wt. %, or less than 0.2 wt. %, or less than 0.1 wt. %, or less than 0.05 wt. %, or less than 0.01 wt. % of Cr, calculated as Cr₂O₃ on a calcined basis.

The present inventors have determined that suitable particulate dehydrogenation catalysts can be made using the P1, P2, M1, M2 and S1 components described herein, e.g., in some embodiments without the use of other promotor or catalytic species. In certain desirable embodiments, the total amount of the primary species (e.g., P1 and P2), promoters (e.g., M1 and M2), and support (e.g., S1) is at least 80 wt. %, or at least 85 wt. %, or at least 90 wt. %, or at least 95 wt. %, or at least 97 wt. %, or at least 98 wt. %, or at least 99 wt. %, or at least 99.5 wt. % of the particulate dehydrogenation catalyst (i.e., P1, P2, M1, and M2 calculated as elemental metal and S1 calculated as oxide on a calcined basis).

In certain desirable embodiments, S1 comprises a covalent network structure, throughout which structure one or more of the primary species (e.g., P1 and P2) and promoters (e.g., M1 and M2) are dispersed.

Conventional methods can be adapted for use in preparing the particulate dehydrogenation catalysts of the disclosure. For example, various hydrolysis-polycondensation, precipitation, and impregnation processes can be used, singly or in combination, to provide the particulate dehydrogenation catalyst. Support materials can suitably be made, for example, by a hydrolysis-polycondensation process of one or more hydroxide or oxy compounds of silicon, aluminum, titanium, and/or zirconium, such as alkoxides (e.g., aluminum isopropoxide, tetraethyl orthosilicaate, titanium n-butoxide, zirconium n-propoxide, etc.), oxynitrates (e.g., zirconyl nitrate, etc.), and hydroxides (e.g., aluminum hydroxide, etc.). For example, in certain embodiments as otherwise described herein, S1 comprises the calcined product of a hydrolysis-polycondensation of one or more oxy compounds of silicon, aluminum, zirconium, and/or titanium (e.g., alkoxides, oxynitrates, and hydroxides). In certain such embodiments, the calcination (e.g., before impregnation) is conducted at a temperature within the range of 500° C. to 1200° C., or 500° C. to 1000° C., or 500° C. to 800° C., or 500° C. to 600° C., or 700° C. to 1200° C., or 900° C. to 1200° C., or 1100° C. to 1200° C., or 600° C. to 800° C., or 700° C. to 900° C., or 800° C. to 1000° C., or 900° C. to 1100° C., or 1000° C. to 1200° C.

Certain of the P1, P2, M1 and M2 species can be formulated together with S1 through hydrolysis-polycondensation. P1, P2, M1 and M2 species can alternatively or additionally be provided to the support through impregnation. Suitable methods for formulating the P1, P2, M1 and/or M2 species together with S1, as well as suitable methods for providing the P1, P2, M1 and/or M2 species to the support through impregnation are generally known in the art.

For example, in certain embodiments, a method for making a particulate dehydrogenation catalyst as described herein includes providing a support S1 (e.g., the product of a hydrolysis-polycondensation reaction of one or more silicon and aluminum oxy compounds), impregnating S1 with P1, P2, M1 and M2 via one or more impregnation steps to provide the desired amounts of P1, P2, M1 and M2 in the final particulate dehydrogenation catalyst. In each such impregnation step, an impregnation solution (e.g., an aqueous impregnation solution) containing one or more of a P1 source, a P2 source, an M1 source, and an M2 source, is contacted with the support. After impregnation, it can be dried and/or calcined. In certain such embodiments, providing S1 comprises reacting one or more S1 sources, e.g., in a hydrolysis-polycondensation reaction, with the S1 sources being one or more oxy compounds, e.g., oxides (e.g., alumina, silica, titania, zirconia), alkoxides (e.g., tetraethyl orthosilicate, aluminum isopropoxide, titanium n-butoxide, zirconium n-propoxide), oxynitrates (e.g., zirconyl nitrate), nitrates, acetylacetonates, or hydroxides (e.g., aluminum hydroxide). The amounts and identities of the various components (e.g., P1, P2, M1, M2, and S1) can be as otherwise described above with respect to the particulate dehydrogenation catalysts of the disclosure (i.e., measured with respect to the final particulate dehydrogenation catalyst).

In another example, in certain embodiments, the method includes reacting an S1 source (e.g., as otherwise described herein) in the presence of one or more of a P1 source, a P2 source, an M1 source, and an M2 source, and calcining the reaction product to provide an silica-alumina support S1 formulated with one or more of P1, P2, M1 and M2. One or more of a P1 source, a P2 source, an M1 source, and an M2 source can then be provided to the calcined reaction product via one or more impregnation steps to provide the desired amounts of P1, P2, M1 and M2 in the final particulate dehydrogenation catalyst (i.e., each coming from being formulated together with the support, added via impregnation, or a combination thereof). The amounts and identities of the various components (e.g., P1, P2, M1, M2, S1) can be as otherwise described above with respect to the particulate dehydrogenation catalyst of the disclosure.

In certain embodiments as otherwise described herein, the method comprises impregnating a support S1 (e.g., including a mixture of silica and alumina) with an impregnation solution comprising a P1 salt (e.g., a gallium salt) to form a P1-formulated (e.g., Ga-formulated) support S1. In other embodiments of the methods as otherwise described herein, the method comprises reacting an S1 source in the presence of a P1 source, for example, by acidifying an aqueous mixture of aluminum hydroxide, silica, and gallium (e.g., in the form of a nitrate, isopropoxide or acetylacetonate) and calcining the reaction product to provide a silica-alumina support S1 formulated with P1 (e.g., Ga).

In certain embodiments as otherwise described herein, the method comprises impregnating a support S1 (e.g., including a mixture of silica and alumina) with an impregnation solution comprising a P2 salt (e.g., a cerium salt and/or a lanthanum salt) to provide a P2-formulated support S1. In other embodiments of the methods as otherwise described herein, the method comprises reacting an S1 source in the presence of a P2 source, for example, by acidifying an aqueous mixture of aluminum hydroxide, silica, gallium (e.g., in the form of a nitrate, isopropoxide or acetylacetonate) and cerium and/or lanthanum (e.g., in the form of isopropoxide, acetylacetonate or nitrate), and calcining the reaction product to provide a support S1 formulated with P2 (e.g., Ce or La).

In certain embodiments as otherwise described herein, the method comprises reacting an S1 source in the presence of a P1 source and a P2 source, for example, by acidifying an aqueous mixture of aluminum hydroxide, silica, cerium and/or lanthanum (e.g., in the form of isopropoxide, oxylate, carbonate, acetylacetonate, ammonia nitrate or nitrate, and its oxides), and calcining the reaction product to provide a support S1 formulated with P1 and P2 (e.g., gallium and cerium and/or lanthanum).

In certain embodiments, a method for preparing a particulate dehydrogenation catalyst as described herein includes providing a support S1 (e.g., including a mixture of silica and alumina) formulated with P1 (e.g., Ga). The formulation with P1 can be through an initial impregnation step, or through reaction of a P1 source together with the S1 source(s). The P1-formulated support S1 can be impregnated with P2, M1 and M2 (e.g., using an impregnation solution comprising a P2 source, an M1 source and an M2 source). In certain such embodiments, when Ce is present in the dehydrogenation catalyst, support is impregnated with one or more of Ba and La. The impregnated material can then be calcined.

In certain embodiments, a method for preparing a particulate dehydrogenation catalyst as described herein includes providing a support S1 (e.g., including a mixture of silica and alumina) formulated with P1 (e.g., Ga) and P2 (e.g., Ce). The formulation with P1 and P2 can be through an initial impregnation step, or through reaction of P1 source and P2 sources together with the S1 source(s). The P1/P2-formulated support S1 can be impregnated with M1 and M2 (e.g., using an impregnation solution comprising an M1 source and an M2 source). In certain such embodiments, the support is impregnated with one or more of Ba and La. The impregnated material can then be calcined.

In certain embodiments as otherwise described herein, the P1 source is a gallium salt, e.g., gallium nitrate, gallium isopropoxide, or gallium acetylacetonate.

In certain embodiments as otherwise described herein, the P2 source is a salt. For example, in certain embodiments, the P2 source is a cerium salt, e.g., cerium nitrate, cerium isopropoxide or cerium acetylacetonate. In another example, in certain embodiments, the P2 source is a lanthanum salt, e.g., cerium nitrate, cerium isopropoxide or cerium acetylacetonate.

In certain embodiments as otherwise described herein, the M1 source is a salt. For example, in certain embodiments, the M1 source is a platinum salt, e.g., Pt(NH₃)₄(NO₃)₂ or H₂PtCl₄. In another example, in certain embodiments of the methods as otherwise described herein, the M1 source is a palladium salt, e.g., Pd(NO₃)₂.

In certain embodiments as otherwise described herein, the M2 source is a salt. For example, in certain embodiments, the M1 source is a salt of a group 1 element, e.g., KNO₃. In another example, in certain embodiments, the M2 source is a salt of a group 2 element, e.g., Mg(NO₃)₂, Ca(NO₃)₂, Sr(NO₃)₂, or Ba(NO₃)₂.

While particular salt species have been described above, the person of ordinary skill in the art will appreciate that other salts and other metallic can be used in the methods described herein.

As described above, the method includes calcining the impregnated support S1. In certain embodiments of the methods as otherwise described herein, the impregnated support S1 is calcined at a temperature within the range of about 500° C. to about 1100° C. For example, in certain embodiments, the impregnated support S1 is calcined at a temperature within the range of about 550° C. to about 1100° C., or about 600° C. to about 1100° C., or about 650° C. to about 1100° C., or about 700° C. to about 1100° C., or about 750° C. to about 1100° C., or about 500° C. to about 1050° C., or about 500° C. to about 1000° C., or about 500° C. to about 950° C., or about 500° C. to about 900° C., or about 500° C. to about 850° C., or about 550° C. to about 1050° C., or about 600° C. to about 1000° C., or about 650° C. to about 950° C.

In certain embodiments as otherwise described herein, the impregnated support S1 is calcined for a period of time within the range of about 5 min. to about 12 hr. For example, in certain embodiments, the impregnated support S1 is calcined for a period of time within the range of about 10 min. to about 12 hr., or about 15 min. to about 12 hr., or about 20 min. to about 12 hr., or about 30 min. to about 12 hr., or about 45 min. to about 12 hr., or about 1 hr. to about 12 hr., or about 1.5 hr. to about 12 hr., or about 2 hr. to about 12 hr., or about 5 min. to about 11 hr., or about 5 min. to about 10 hr., or about 5 min. to about 9 hr., or about 5 min. to about 8 hr., or about 5 min. to about 7.5 hr., or about 5 min. to about 7 hr., or about 5 min. to about 6.5 hr., or about 5 min. to about 6 hr., or about 5 min. to about 5.5 hr., or about 5 min. to about 5 hr., or about 30 min. to about 11 hr., or about 1 hr. to about 10 hr., or about 1.5 hr. to about 9 hr., or about 2 hr. to about 8 hr.

In certain embodiments as otherwise described herein, the impregnated support S1 is dried before calcination. In certain embodiments, the impregnated support S1 is dried at a temperature within the range of about 80° C. to about 240° C. For example, in certain embodiments, the impregnated support S1 is dried at a temperature within the range of about 80° C. to about 220° C., or about 80° C. to about 200° C., or about 80° C. to about 180° C., or about 100° C. to about 240° C., or about 120° C. to about 240° C., or about 140° C. to about 240° C., or about 100° C. to about 220° C., or about 120° C. to about 200° C., or about 140° C. to about 180° C.

In certain embodiments as otherwise described herein, the impregnated support S1 is dried for a period of time within the range of about 4 hr. to about 36 hr. For example, in certain embodiments, the impregnated support S1 is dried for a period of time within the range of about 4 hr. to about 30 hr., or about 4 hr. to about 24 hr., or about 4 hr. to about 22 hr., or about 4 hr. to about 20 hr., or about 6 hr. to about 36 hr., or about 8 hr. to about 36 hr., or about 10 hr. to about 36 hr., or about 12 hr. to about 36 hr., or about 6 hr. to about 30 hr., or about 8 hr. to about 24 hr., or about 10 hr. to about 22 hr., or about 12 hr. to about 20 hr.

In certain embodiments as otherwise described herein, the average particle size of the particulate dehydrogenation catalyst is within the range of 5 μm to 4 mm. For example, in certain such embodiments, the average particle size of the particulate dehydrogenation catalyst is within the range of 5 μm to 3 mm, or 5 μm to 2 mm, or 5 μm to 1 mm, or 5 μm to 750 μm, or 5 μm to 500 μm, or 5 μm to 250 μm, or 5 μm to 100 μm, or 100 μm to 4 mm, or 250 μm to 4 mm, or 500 μm to 4 mm, or 750 μm to 4 mm, or 1 mm to 4 mm, or 1.5 mm to 4 mm, or 2 mm to 4 mm, or 3 mm to 4 mm, or 100 μm to 500 mm, or 250 μm to 1 mm, or 500 μm to 1.5 mm, or 1 mm to 2 mm, or 2 mm to 3 mm.

In certain embodiments as otherwise described herein, the particulate dehydrogenation catalyst comprises at least 65 wt. % of the mixed-bed system. For example, in certain embodiments as otherwise described herein, the particulate dehydrogenation catalyst comprises at least 70 wt. %, or at least 75 wt. %, or at least 80 wt. %, or at least 90 wt. % of the mixed-bed system.

As described above, the particulate non-catalytic additive includes a heat-generating material. As used herein, heat-generating materials such as, for example, metal oxide heat-generating materials, are catalytically inert components (e.g., substantially inert with respect to alkane dehydrogenation) that generate heat upon exposure to oxidizing and/or reducing reaction conditions (e.g., regeneration and/or reduction). For example, in certain embodiments as otherwise described herein, the heat-generating material includes copper oxide, copper aluminate, calcium sulfate, copper sulfate, zinc oxide, nickel oxide, iron oxide, tin oxide, cobalt oxide, vanadium oxide, lanthanum oxide, cerium oxide, manganese oxide, and any mixture thereof.

This heat may be generated and immediately provided to a dehydrogenation reaction. For example, an oxidized metal oxide heat-generating material (e.g., copper(II) oxide, copper(I) oxide), exposed to the reducing environment of a dehydrogenation reactor operating under reaction conditions, can provide heat to dehydrogenation reactions occurring in the reactor (e.g., dehydrogenation of propane to propylene).

Of course, heat may also be generated and stored in the material of the catalyst for a subsequent dehydrogenation reaction. For example, a reduced metal oxide heat-generating material (e.g., copper(0), copper(I) oxide), exposed to the oxidizing environment of a dehydrogenation reactor operating under regeneration conditions, can provide heat to the material of the mixed-bed system that will help to drive subsequent dehydrogenation reactions upon returning to reaction conditions.

A wide variety of metal oxide heat-generating materials can be present in the particulate non-catalytic additive. For example, in certain embodiments as otherwise described herein, the metal oxide comprises copper, chromium, molybdenum, vanadium, cerium, zinc, nickel, iron, tungsten, manganese, lanthanum, or any mixture thereof. For example, in certain desirable embodiments as otherwise described herein, the particulate non-catalytic additive comprises a copper oxide heat-generating material, which is substantially inert with respect to alkane dehydrogenation (e.g., less than 20%, or less than 10% of the catalytic activity of the particulate dehydrogenation catalyst), but can desirably generate heat upon reduction (e.g., to Cu(0)), and subsequently upon oxidation (e.g., back to CuO). As used herein, a metal oxide heat-generating material may, at a given point, be in an oxidized form or a reduced form.

As described above, the non-catalytic additive includes a carrier selected from inorganic oxides, clays, and any mixture thereof. In addition to acting as a carrier for a heat-generating material, such materials can desirably add thermal mass to the mixed-bed system to provide more heat capacity for heat generated by the heat-generating material.

Accordingly, in certain embodiments as otherwise described herein, the particulate non-catalytic additive comprises a metal oxide heat-generating material, present in an amount within the range of 0.5 wt. % to 60 wt. %. In certain such embodiments, the heat-generating material is present in the particulate non-catalytic additive in an amount within the range of 0.5 wt. % to 55 wt. %, or 0.5 wt. % to 50 wt. %, or 0.5 wt. % to 45 wt. %, or 0.5 wt. % to 40 wt. %, or 0.5 wt. % to 35 wt. %, or 0.5 wt. % to 30 wt. %, or 0.5 wt. % to 25 wt. %, or 1 wt. % to 60 wt. %, or 2.5 wt. % to 60 wt. %, or 5 wt. % to 60 wt. %, or 10 wt. % to 60 wt. %, or 15 wt. % to 60 wt. %, or 20 wt. % to 60 wt. %, or 25 wt. % to 60 wt. %, or 30 wt. % to 60 wt. %, or 1 wt. % to 55 wt. %, or 2.5 wt. % to 50 wt. %, or 5 wt. % to 45 wt. %.

For example, in certain embodiments as otherwise described herein, the particulate non-catalytic additive comprises copper oxide (e.g., present in the particulate non-catalytic additive in an amount within the range of 3 wt. % to 20 wt. %). In certain such embodiments, the particulate non-catalytic additive comprises manganese oxide (e.g., present in the particulate non-catalytic additive in an amount of up to 5 wt. %) and/or cerium oxide (e.g., present in the particulate non-catalytic additive in an amount of up to 10 wt. %).

In certain embodiments as otherwise described herein, the carrier is present in the particulate non-catalytic additive in an amount within the range of 40 wt. % to 99 wt. %. For example, in certain embodiments as otherwise described herein, the carrier includes (e.g., is) one or more inorganic oxides such as, for example, silica, alumina (e.g., α-, γ-, η-, θ-, X, κ-, and δ-alumina), aluminate (e.g., Ca-aluminate, Zn-aluminate, and Mg-aluminate), or any mixture thereof (e.g., a mixture of silica and alumina). In another example, in certain such embodiments, the carrier includes (e.g., is) one or more clays such as, for example, kaolin. In certain embodiments as otherwise described herein, the carrier includes (e.g., is) one or more components selected from inorganic oxides (e.g., silica, alumina, calcium aluminate, or a mixture thereof) and clay (e.g., kaolin), present in a combined amount within the range of 40 wt. % to 99 wt. %, or 40 wt. % to 95 wt. %, or 40 wt. % to 90 wt. %, or 40 wt. % to 80 wt. %, or 40 wt. % to 70 wt. %, or 40 wt. % to 60 wt. %, or 50 wt. % to 99 wt. %, or 60 wt. % to 99 wt. %, or 45 wt. % to 90 wt. %, or 50 wt. % to 80 wt., or 55 wt. % to 75 wt. %.

For example, in certain embodiments as otherwise described herein, the particulate non-catalytic additive comprises the carrier, present in a combined amount within the range of 55 wt. % to 95 wt. %, and the metal oxide, present in a combined amount within the range of 5 wt. % to 45 wt. %. In certain such embodiments, the carrier is alumina and/or calcium aluminate. In certain such embodiments, the metal oxide is copper oxide.

In certain embodiments as otherwise described herein, the heat-generating material and carrier comprise at least 70 wt. %, or at least 75 wt. %, or at least 80 wt. %, or at least 85 wt. %, or at least 90 wt. %, or at least 95 wt. %, or at least 97.5 wt. %, or at least 98 wt. %, or at least 99 wt. % of the particulate non-catalytic additive. For example, in certain embodiments as otherwise described herein, the particulate non-catalytic additive includes a copper oxide heat-generating material and one or more components selected from inorganic oxides, present in a combined amount of at least 70 wt. %, or at least 80 wt. %, or at least 90 wt. % of the particulate non-catalytic additive.

In certain embodiments as otherwise described herein, the average particle size of the particulate non-catalytic additive is within the range of 5 μm to 5 mm. For example, in certain such embodiments, the average particle size of the non-catalytic additive is within the range of 5 μm to 4 mm, or 5 μm to 3 mm, or 5 μm to 2 mm, or 5 μm to 1 mm, or 5 μm to 750 μm, or 5 μm to 500 μm, or 5 μm to 250 μm, or 5 μm to 100 μm, or 100 μm to 5 mm, or 250 μm to 5 mm, or 500 μm to 5 mm, or 750 μm to 5 mm, or 1 mm to 5 mm, or 1.5 mm to 5 mm, or 2 mm to 5 mm, or 3 mm to 5 mm, or 100 μm to 500 mm, or 250 μm to 1 mm, or 500 μm to 1.5 mm, or 1 mm to 2 mm, or 2 mm to 3 mm, or 3 mm to 4 mm, or 4 mm to 5 mm.

In certain embodiments as otherwise described herein, the particulate non-catalytic additive is present in the mixed-bed system in an amount within the range of 0.5 wt. % to 30 wt. %. In certain such embodiments, the non-catalytic additive is present in the mixed-bed system in an amount within the range of 1 wt. % to 30 wt. %, or 2.5 wt. % to 30 wt. %, or 5 wt. % to 30 wt. %, or 10 wt. % to 30 wt. %, or 0.5 wt. % to 25 wt. %, or 0.5 wt. % to 20 wt. %, or 0.5 wt. % to 15 wt. %, or 0.5 wt. % to 10 wt. %, or 1 wt. % to 25 wt. %, or 1 wt. % to 15 wt. %, or 2.5 wt. % to 10 wt. %.

In certain embodiments as otherwise described herein, the ratio of particulate dehydrogenation catalyst and particulate non-catalytic additive present in the mixed-bed system is 99:1 to 3:2 by weight. For example, in certain embodiments, the ratio of particulate dehydrogenation catalyst and particulate non-catalytic additive present in the mixed-bed system is 99:1 to 1:1, or 99:1 to 2:3, or 99:1 to 3:7, or 99:1 to 1:4, or 99:1 to 1:9, or 49:1 to 3:2, or 97:3 to 3:2, or 19:1 to 3:2, or 9:1 to 3:2, or 4:1 to 3:2, or 19:1 to 1:1, or 9:1 to 2:3.

In certain embodiments as otherwise described herein, the particulate dehydrogenation catalyst and particulate non-catalytic additive are present in the system in a combined amount of at least 90 wt. %. For example, in certain embodiments, the particulate dehydrogenation catalyst and particulate non-catalytic additive are present in the system in a combined amount of at least 92.5 wt. %, or at least 95 wt. %, or at least 97.5 wt. %, or at least 98 wt. %, or at least 99 wt. %.

The particulate non-catalytic additives described herein may be prepared by conventional procedures, as would be understood by the person of ordinary skill in the art. For example, in certain embodiments, impregnation techniques are used to provide heat-generating materials to the particulate non-catalytic additive.

Advantageously, the present inventors have determined that the use of mixed-bed systems described herein can catalyze a hydrocarbon dehydrogenation reaction at an efficiency comparable to or better than conventional, commercially available catalyst materials. Accordingly, another aspect of the disclosure is a method for dehydrogenating alkanes that includes providing a mixed-bed system as described herein, the system comprising an oxidized heat-generating material, and then contacting a hydrocarbon feed with the system under conditions sufficient to cause hydrocarbon dehydrogenation. One particular aspect relates to a method for dehydrogenating hydrocarbons, the method comprising contacting a hydrocarbon feed with a mixed-bed system as described herein; and performing a plurality of reaction cycles, each reaction cycle comprising contacting a hydrocarbon feed with the system to dehydrogenate the hydrocarbon feed and to form a deactivated system comprising a reduced heat-generating material; and contacting the deactivated system with an oxygen-containing gas (e.g., air) to oxidize the heat-generating material.

As noted above, the heat-generating material comprising the particulate non-catalytic additive can generate heat upon exposure to oxidizing and/or reducing reaction conditions (e.g., regeneration and/or reduction). Accordingly, in certain embodiments as otherwise described herein, reduction of the heat-generating material provides heat to the system. In certain embodiments as otherwise described herein, oxidation of the heat-generating material provides heat to the system. In certain embodiments, reduction of the heat-generating material provides heat to the system and oxidation of the heat-generating material provides heat to the system.

In certain embodiments as otherwise described herein, at least 50 wt. %, or at least 75 wt. %, or at least 80 wt. %, or at least 85 wt. %, or at least 90 wt. %, or at least 95 wt. %, or at least 97.5 wt. % of the oxidized heat-generating material comprises a metal having an oxidized oxidation state (e.g., copper(I) oxide, or copper(II) oxide).

In certain embodiments as otherwise described herein, the temperature of the provided mixed-bed system is within the range of 400° C. to 1200° C. For example, in certain embodiments as otherwise described herein, the temperature of the provided mixed-bed system is within the range of 400° C. to 700° C., or 400° C. to 650° C., or 400° C. to 600° C., or 400° C. to 550° C., or 450° C. to 750° C., or 500° C. to 750° C., or 550° C. to 750° C., or 600° C. to 750° C., or 450° C. to 700° C., or 500° C. to 650° C.

In certain embodiments as otherwise described herein, the hydrocarbon feed comprises one or more C₃-C₅ alkanes. For example, in certain embodiments, the hydrocarbon feed comprises propane.

The contacting of the feed with the mixed-bed systems described herein can be conducted in a variety of ways familiar to the person of ordinary skill in the art. Conventional equipment and processes can be used in conjunction with the mixed-bed systems of the disclosure to provide beneficial performance. Thus, the mixed-bed system may be contained in one bed within a reactor vessel or divided up among a plurality of beds within a reactor. The reaction system may contain one or more reaction vessels in series. The feed to the reaction zone can flow vertically upwards, or downwards through the catalyst bed in a typical plug flow reactor, or horizontally across the catalyst bed in a radial flow type reactor.

For example, in certain embodiments as otherwise described herein, the mixed-bed system comprises an intimate mixture (i.e., a substantially evenly distributed mixture) of the particulate dehydrogenation catalyst and particulate non-catalytic additive contained in one or more reactor beds. In another example, in certain embodiments as otherwise described herein, the mixed-bed system comprises adjacent layers (i.e., substantially separate distributions) of the particulate dehydrogenation catalyst and particulate non-catalytic additive contained in one or more reactor beds. For example, in certain desirable embodiments, at least a portion (e.g., all) of the particulate non-catalytic additive of the mixed-bed system comprises a layer over the particulate dehydrogenation catalyst of the mixed-bed system, such that the hydrocarbon feed introduced into a reactor first contacts the layer comprising particulate non-catalytic additive.

The contacting of the feed with the mixed-bed system can be performed using conventional methods. For example, the feed may be introduced into a reaction zone containing the mixed-bed system at a constant rate, or alternatively, at a variable rate.

In certain embodiments as otherwise described herein, the feed is contacted with the mixed-bed system at a liquid hourly space velocity (LHSV) within the range of 0.5 h⁻¹ to 4 h⁻¹. For example, in certain embodiments as otherwise described herein, the feed is contacted with the mixed-bed system at a liquid hourly space velocity of 0.75 h⁻¹ to 4 h⁻¹, or 1 h⁻¹ to 4 h⁻¹, or 1.25 h⁻¹ to 4 h⁻¹, or 1.5 h⁻¹ to 4 h⁻¹, or 0.5 h⁻¹ to 3.75 h⁻¹, or 0.5 h⁻¹ to 3.5 h⁻¹, or 0.5 h⁻¹ to 3.25 h⁻¹, or 0.5 h⁻¹ to 3 h⁻¹, or 0.5 h⁻¹ to 2.75 h⁻¹, or 0.5 h⁻¹ to 2.5 h⁻¹, or 0.75 h⁻¹ to 3.5 h⁻¹, or 1 h⁻¹ to 3 h⁻¹, or 1.25 h⁻¹ to 2.75 h⁻¹, or 1.5 h⁻¹ to 2.5 h⁻¹.

In certain embodiments as otherwise described herein, the contacting of the feed is carried out at a temperature within the range of 400° C. to 750° C. For example, in certain embodiments as otherwise described herein, the contacting of the feed with the mixed-bed system is carried out at a temperature within the range of 400° C. to 700° C., or 400° C. to 650° C., or 400° C. to 600° C., or 400° C. to 550° C., or 450° C. to 750° C., or 500° C. to 750° C., or 550° C. to 750° C., or 600° C. to 750° C., or 450° C. to 700° C., or 500° C. to 650° C.

In certain embodiments as otherwise described herein, the contacting of the feed is carried out at a pressure within the range of 0.1 bar to 1 bar. For example, in certain embodiments as otherwise described herein, the contacting of the feed is carried out at a pressure within the range of 0.1 bar to 0.9 bar, or 0.1 bar to 0.8 bar, or 0.1 bar to 0.7 bar, or 0.1 bar to 0.6 bar, or 0.1 bar to 0.5 bar, or 0.2 bar to 1 bar, or 0.3 bar to 1 bar, or 0.4 bar to 1 bar, or 0.5 bar to 1 bar, or 0.2 bar to 0.9 bar, or 0.3 bar to 0.8 bar, or 0.4 bar to 0.7 bar.

The present inventors note that as the hydrocarbon feed is contacted with the mixed-bed system (e.g., as the feed continues to flow through a fixed-bed reactor), residual hydrocarbons and reaction by-products (e.g., coke) are adsorbed onto the surface of the mixed-bed system particles and the heat-generating material is reduced, providing a deactivated mixed-bed system. In certain embodiments as otherwise described herein, at least 50 wt. %, or at least 75 wt. %, or at least 80 wt. %, or at least 85 wt. %, or at least 90 wt. %, or at least 95 wt. %, or at least 97.5 wt. % of the reduced heat-generating metal species (i.e., comprising the deactivated mixed-bed system) has a reduced oxidation state (e.g., +1 as in copper(I) oxide, or 0 as in copper (0)).

In certain embodiments as otherwise described herein, each reaction cycle includes contacting the deactivated mixed-bed system with steam or inert gas before contacting the oxygen-containing gas. The present inventors note that as the steam or inert gas is contacted with the mixed-bed system, residual hydrocarbons are removed from the surface of the mixed-bed system particles, providing a stripped deactivated mixed-bed system.

The present inventors note that as the oxygen-containing gas is contacted with the (e.g., stripped) deactivated mixed-bed system, reaction by-products (e.g., coke) are removed from the surface of mixed-bed system particles and the heat-generating material is oxidized, providing an activated mixed-bed system.

In certain embodiments as otherwise described herein, at least 50 wt. %, or at least 75 wt. %, or at least 80 wt. %, or at least 85 wt. %, or at least 90 wt. %, or at least 95 wt. %, or at least 97.5 wt. % of the oxidized heat-generating metal species (i.e., comprising the activated mixed-bed system) has an oxidized oxidation state (e.g., +2 as in copper(II) oxide, or +1 as in copper(I) oxide).

In certain embodiments as otherwise described herein, the temperature of the oxygen-containing gas is within the range of 400° C. to 750° C. For example, in certain embodiments as otherwise described herein, the temperature of the air is within the range of 400° C. to 700° C., or 400° C. to 650° C., or 400° C. to 600° C., or 400° C. to 550° C., or 450° C. to 750° C., or 500° C. to 750° C., or 550° C. to 750° C., or 600° C. to 750° C., or 450° C. to 700° C., or 500° C. to 650° C.

### EXAMPLES

The Examples that follow are illustrative of specific embodiments of the invention, and various uses thereof. They are set forth for explanatory purposes only, and are not to be taken as limiting the invention.

### Example 1. Catalyst Preparation

Particulate dehydrogenation catalyst A was made by impregnation of 30 g calcined Siralox10 carrier with an aqueous solution containing 5.1 g Ga(NO₃)₃, 0.0018 g Pt(NH₃)₄(NO₃)₂, 1.10 g Ce(NO₃)₃.6H₂O, 0.21 g KNO₃, and 0.55 g Ba(NO₃)₂ and 15.5 g DI-water by incipient wetness. The catalyst was dried at 120° C. for 16 hours and calcined at 750° C. in air for 2 hours.

A comparative alumina-supported particulate dehydrogenation catalyst C was prepared according to conventional methods.

TABLE 1
Particulate dehydrogenation catalysts

| | SiO₂ | Al₂O₃ | Ga | Ce | Pt | K | Ba |
|---|---|---|---|---|---|---|---|
| Cat. | (wt. %) | (wt. %) | (wt. %) | (wt. %) | (ppm) | (wt. %) | (wt. %) |
| A | 10 | 90 | 4.5 | 1.0 | 25 | 0.25 | 1 |

20 cc particulate dehydrogenation catalyst A was placed into a fixed-bed reactor, and 5 g of a particulate non-catalytic additive (7 wt. % copper oxide on calcium aluminate or α-alumina) was layered over the particulate dehydrogenation catalyst to provide mixed-bed system A-HGM.

20 cc of particulate dehydrogenation catalyst A was placed into a fixed-bed reactor to provide comparative system C-1.

20 cc of particulate dehydrogenation catalyst C was placed into a fixed-bed reactor to provide comparative system C-2.

20 cc of particulate dehydrogenation catalyst C was placed into a fixed-bed reactor, and 5 g of a particulate non-catalytic additive containing copper oxide was layered over the particulate dehydrogenation catalyst to provide comparative system C-HGM.

### Example 2. Propane Dehydrogenation

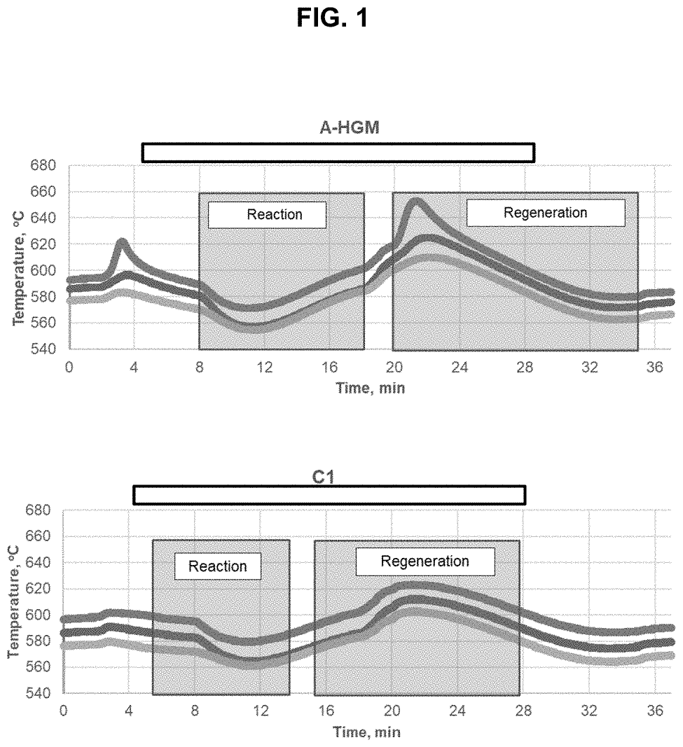

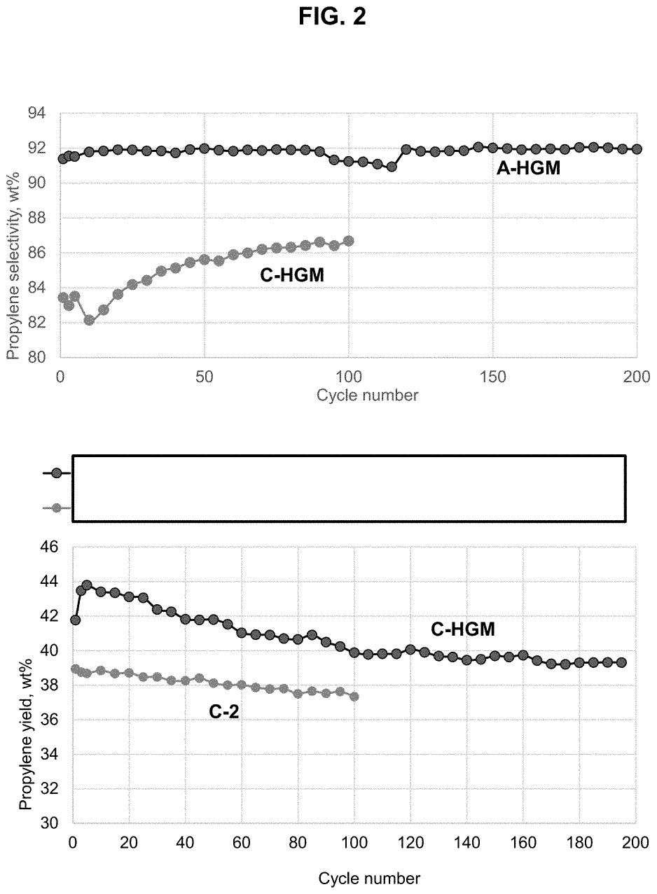

Catalyst-comprising systems prepared according to Example 1 were tested as prepared. A feed containing 100 mol. % propane was passed over a catalyst bed at a total pressure of 0.5 atm., at 1.5 h⁻¹ liquid hourly space velocity (LHSV) in cyclic mode, where 10 minutes of propane dehydrogenation is followed by catalyst regeneration in air. Results are provided in Table 2 below. Profiles of catalyst bed temperatures, propylene yield, and propylene selectivity are shown in FIGS. 1-2.

TABLE 2
Propane Dehydrogenation

| | | | Conversion | Selectivity | Yield |
|---|---|---|---|---|---|
| Sys. | Cycle | T (° C.) | (wt. %) | (wt. %) | (wt. %) |
| A-HGM | 1 | 568 | 45.7 | 91.4 | 41.8 |
| A-HGM | 100 | 568 | 43.7 | 91.2 | 39.9 |
| C-1 | 1 | 568 | 47.0 | 92.3 | 43.4 |
| C-1 | 100 | 573 | 44.3 | 92.3 | 40.9 |
| C-2 | 1 | 571 | 49.6 | 83.4 | 41.1 |
| C-2 | 100 | 574 | 44.9 | 86.7 | 38.9 |
| C-HGM | 1 | 563 | 48.7 | 80.0 | 38.96 |
| C-HGM | 100 | 567 | 43.2 | 86.4 | 37.3 |

The results show that mixed-bed system A-HGM provided yields comparable to or better than those of chromium-based systems, with or without a particulate non-catalytic additive. Additionally, the results highlight the improved energy efficiency of system A-HGM relative to that of a comparative system including the same particulate dehydrogenation catalyst, but lacking a particulate non-catalytic additive—after 100 cycles, the temperature necessary to maintain a desirable propylene yield was 5° C. lower than that of system C-1. This can result, over time, in a reduced heat consumption and also a reduced rate of degradation of the catalyst. The foregoing detailed description and the accompanying drawings have been provided by way of explanation and illustration, and are not intended to limit the scope of the appended claims. Many variations in the presently preferred embodiments illustrated herein will be apparent to one of ordinary skill in the art, and remain within the scope of the appended claims and their equivalents.

It is to be understood that the elements and features recited in the appended claims may be combined in different ways to produce new claims that likewise fall within the scope of the present disclosure. Thus, whereas the dependent claims appended below depend from only a single independent or dependent claim, it is to be understood that these dependent claims can, alternatively, be made to depend in the alternative from any preceding claim—whether independent or dependent—and that such new combinations are to be understood as forming a part of the present specification.

Various aspects and embodiments of the present disclosure are set out in the following embodiments, which can be combined in any number and in any combination that is not technically or logically inconsistent:

Embodiment 1. A mixed-bed system comprising
a particulate dehydrogenation catalyst comprising a primary species P1 selected from Ga, In, TI, Ge, Sn, Pb, and any mixture thereof as an active metal, disposed on a support; and
a particulate non-catalytic additive comprising a heat-generating material and a carrier selected from inorganic oxides, clays, and any mixture thereof.
   Embodiment 2. The system of embodiment 1, wherein P1 is selected from Ga, Ge, In, Sn, TI, and any mixture thereof.
   Embodiment 3. The system of embodiment 1, wherein P1 is Ga.
   Embodiment 4. A mixed-bed system comprising
   a particulate dehydrogenation catalyst comprising
a primary species P1 selected from Ga, In, TI, Ge, Sn, Pb, and any mixture thereof, present in the particulate dehydrogenation catalyst in an amount within the range of 0.05 wt. % to 20 wt. %, calculated as elemental metal on a calcined basis;
a primary species P2 selected from the lanthanides and any mixture thereof, present in the particulate dehydrogenation catalyst in an amount within the range of 0.05 wt. % to 10 wt. %, calculated as elemental metal on a calcined basis;
a promoter M1 selected from Ni, Pd, Pt, La, Ir, Zn, Fe, Rh, Ru, Mn, Co, W, and any mixture thereof, present in the particulate dehydrogenation catalyst in an amount within the range of 1 ppm to 500 ppm, calculated as elemental metal on a calcined basis;
a promoter M2 selected from Li, Na, K, Rb, Cs, Be, Mg, Ca, Sr, Ba, and any mixture thereof, present in the particulate dehydrogenation catalyst in an amount within the range of 0.05 wt. % to 3 wt. %, calculated as elemental metal on a calcined basis; and
a support S1 selected from silica, alumina, zirconia, titania, yttria and any mixture thereof, present in the particulate dehydrogenation catalyst in an amount within the range of 50 wt. % to 99 wt. %, calculated as oxide on a calcined basis; and
   a particulate non-catalytic additive comprising
a heat-generating material; and
a carrier selected from inorganic oxides, clays, and any mixture thereof.
   Embodiment 5. The system of any of embodiments 1-4, wherein P1 is selected from Ga, Ge, In, Sn, TI, and any mixture thereof.
   Embodiment 6. The system of any of embodiments 1-4, wherein P1 is a mixture of Ga and Sn.
   Embodiment 7. The system of any of embodiments 1-4, wherein P1 is Ga.
   Embodiment 8. The system of any of embodiments 1-7, wherein P1 is present in the particulate dehydrogenation catalyst in an amount within the range of 0.1 wt. % to 10 wt. % (e.g., 0.5 wt. % to 10 wt. %, or 2.5 wt. % to 5 wt. %), calculated as elemental metal on a calcined basis.
   Embodiment 9. The system of any of embodiments 4-8, wherein P2 is selected from La, Ce, Nd, and any mixture thereof.
   Embodiment 10. The system of any of embodiments 4-8, wherein P2 is Ce.
   Embodiment 11. The system of any of embodiments 4-8, wherein P2 is a mixture of La and Ce.
   Embodiment 12. The system of any of embodiments 4-11, wherein P2 is present in the particulate dehydrogenation catalyst in an amount within the range of 0.1 wt. % to 6 wt. % (e.g., 0.5 wt. % to 6 wt. %, or 1 wt. % to 4 wt. %), calculated as elemental metal on a calcined basis.
   Embodiment 13. The system of any of embodiments 4-12, wherein M1 is selected from Pt, Ir, La, Zn, Fe, Rh, Pd, Ru, and any mixture thereof.
   Embodiment 14. The system of any of embodiments 4-12, wherein M1 is selected from Pd, Pt, Ir, La, and any mixture thereof.
   Embodiment 15. The system of any of embodiments 4-12, wherein M1 is selected from Pd, Pt, and any mixture thereof.
   Embodiment 16. The system of any of embodiments 4-12, wherein M1 is Pt.
   Embodiment 17. The system of any of embodiments 4-12, wherein M1 is Pd.
   Embodiment 18. The system of any of embodiments 4-17, wherein M1 is present in the particulate dehydrogenation catalyst in an amount within the range of 1 ppm to 500 ppm (e.g., 50 ppm to 400 ppm), calculated as elemental metal on a calcined weight basis.
   Embodiment 19. The system of any of embodiments 4-18, wherein M2 is selected from K, Na, Ce, Li, Ca, Mg, Sr, Ba, and any mixture thereof.
   Embodiment 20. The system of any of embodiments 4-18, wherein M2 is selected from Li, Na, K, Cs, Ba, and any mixture thereof.
   Embodiment 21. The system of any of embodiments 4-18, wherein M2 is K.
   Embodiment 22. The system of any of embodiments 4-18, wherein M2 is a mixture of K and Ba.
   Embodiment 23. The system of any of embodiments 4-22, wherein M2 is present in the particulate dehydrogenation catalyst in an amount within the range of 0.05 wt. % to 2.5 wt. % (e.g., 0.25 wt. % to 2.5 wt. %), calculated as elemental metal on a calcined basis.
   Embodiment 24. The system of embodiment 4, wherein
P1 is selected from Ga, In, TI, Ge, Sn, Pb, and any mixture thereof;
P2 is selected from the lanthanides and any mixture thereof;
M1 is selected from Ni, Pd, Pt, and any mixture thereof;
M2 is selected from Li, Na, K, Rb, Cs, Be, Mg, Ca, Sr, Ba; and;
S1 is a mixture of silica and alumina.
   Embodiment 25. The system of embodiment 4, wherein
P1 is Ga, present in the particulate dehydrogenation catalyst in an amount within the range of 0.1 wt. % to 10 wt. % (e.g., 2.5 wt. % to 5 wt. %), calculated as elemental metal on a calcined basis;
P2 is Ce, present in the particulate dehydrogenation catalyst in an amount within the range of 0.1 wt. % to 6 wt. % (e.g., 1 wt. % to 4 wt. %), calculated as elemental metal on a calcined basis; and
M1 is Pt, present in the particulate dehydrogenation catalyst in an amount within the range of 1 ppm to 500 ppm (e.g., 50 ppm to 400 ppm), calculated as elemental metal on a calcined weight basis.
   Embodiment 26. The system of embodiment 4, wherein
P1 is Ga, present in the particulate dehydrogenation catalyst in an amount within the range of 0.1 wt. % to 10 wt. % (e.g., 2.5 wt. % to 5 wt. %), calculated as elemental metal on a calcined basis;
P2 is a mixture of Ce and La, present in the particulate dehydrogenation catalyst in an amount within the range of 0.1 wt. % to 6 wt. % (e.g., 1 wt. % to 4 wt. %), calculated as elemental metal on a calcined basis; and
M1 is Pt, present in the particulate dehydrogenation catalyst in an amount within the range of 1 ppm to 500 ppm (e.g., 50 ppm to 400 ppm), calculated as elemental metal on a calcined weight basis.
   Embodiment 27. The system of embodiment 25 or embodiment 26, wherein M2 is K, present in the particulate dehydrogenation catalyst in an amount within the range of 0.05 wt. % to 2.5 wt. % (e.g., 0.25 wt. % to 2.5 wt. %), calculated as elemental metal on a calcined basis.
   Embodiment 28. The system of embodiment 18 or embodiment 19, wherein M2 is a mixture of K and Ba, present in the particulate dehydrogenation catalyst in an amount within the range of 0.05 wt. % to 2.5 wt. % (e.g., 0.25 wt. % to 2.5 wt. %), calculated as elemental metal on a calcined basis.
   Embodiment 29. The system of any of embodiments 4-28, wherein P1 includes Ga, and wherein if P2 includes Ce, the particulate dehydrogenation catalyst includes one or more of La and Ba.
   Embodiment 30. The system of any of embodiments 4-28, wherein P1 includes Ga, P2 includes Ce, and the particulate dehydrogenation catalyst includes one or more of La and Ba.
   Embodiment 31. The system of any of embodiments 4-30, wherein S1 includes a mixture of silica and alumina.
   Embodiment 32. The system of embodiment 31, wherein the amount of silica present in S1 is within the range of 1 wt. % to 70 wt. % of S1.
   Embodiment 33. The system of embodiment 31, wherein the amount of silica present in S1 is within the range of 1 wt. % to 50 wt. % of S1.
   Embodiment 34. The system of any of embodiments 31-33, wherein the amount of alumina present in S1 is within the range of 30 wt. % to 99 wt. % (e.g., 50 wt. % to 99 wt. %) of S1.
   Embodiment 35. The system of any of embodiments 31-34, wherein the total amount of alumina and silica present in S1 is at least 80 wt. % (e.g., at least 85 wt. %, at least 90 wt. %, at least 95 wt. %, at least 97.5 wt. %, or at least 99 wt. %) of S1.
   Embodiment 36. The system of any of embodiments 4-30, wherein S1 includes zirconia (e.g., at least 50 wt. % zirconia).
   Embodiment 37. The system of embodiment 36, wherein the amount of zirconia present in S1 is within the range of 50 wt. % to 99 wt. % (e.g., 80 wt. % to 99 wt. %, or 50 wt. % to 75 wt. %) of S1.
   Embodiment 38. The system of embodiment 36 or 37, wherein S1 includes titania (e.g., 25 wt. % to 50 wt. % titania).
   Embodiment 39. The system of any of embodiments 36-38, wherein S1 includes zirconia, alumina, and silica, present in a total amount of at least 80 wt. % (e.g., at least 85 wt. %, at least 90 wt. %, at least 95 wt. %, at least 97.5 wt. %, or at least 99 wt. %) of S1.
   Embodiment 40. The system of any of embodiments 36-38, wherein the total amount of silica and alumina present in S1 is less than 5 wt. % (e.g., less than 4 wt. %, or less than 2 wt. %, or less than 0.5 wt. %, or less than 0.25 wt. %) of S1.
   Embodiment 41. The system of any of embodiments 4-40, wherein S1 is present in the particulate dehydrogenation catalyst in an amount within the range of 50 wt. % to 99 wt. % (e.g., 70 wt. % to 99 wt. %, or 90 wt. % to 99 wt. %).
   Embodiment 42. The system of any of embodiments 4-41, wherein the total amount of P1, P2, M1, M2, and S1 is at least 80 wt. % (e.g., at least 85 wt. %, at least 90 wt. %, at least 95 wt. %, at least 97 wt. %, or at least 98 wt. %, or at least 99 wt. %, or at least 99.5 wt. %) of the particulate dehydrogenation catalyst.
   Embodiment 43. The system of any of embodiments 4-42, wherein S1 comprises a covalent network structure.
   Embodiment 44. The system of embodiment 43, wherein S1 comprises the product of a hydrolysis-polycondensation of one or more oxy compounds of silicon, aluminum, titanium, and/or zirconium (e.g., alkoxides, oxynitrates, and hydroxides).
   Embodiment 45. The system of embodiment 30, wherein S1 comprises the calcined (e.g., at a temperature within the range of 500° C. to 1200° C.) product of a hydrolysis-polycondensation of one or more oxy compounds of silicon, aluminum, titanium, and/or zirconium (e.g., alkoxides, oxynitrates, and hydroxides).
   Embodiment 46. The system of any of embodiments 4-45, wherein the average particle size of the particulate dehydrogenation catalyst is within the range of 5 μm to 4 mm (e.g., 5 μm to 1 mm, or 2 mm to 4 mm).
   Embodiment 47. The system of any of embodiments 1-46, wherein the particulate dehydrogenation catalyst is present in the mixed-bed system in an amount within the range of 65 wt. % to 99.5 wt. % (e.g., 75 wt. % to 99 wt. %).
   Embodiment 48. The system of any of embodiments 1-47, where the particulate dehydrogenation catalyst comprises the calcined (e.g., at a temperature within the range of 500° C. to 1100° C.) product of impregnation of S1 with an impregnation solution (e.g., an aqueous impregnation solution) containing one or more of a P1 source, a P2 source, an M1 source, and an M2 source.
   Embodiment 49. The system of any of embodiments 1-48, wherein the particulate non-catalytic additive comprises a metal oxide heat-generating material, present in an amount within the range of 0.5 wt. % to 60 wt. % (e.g., 1 wt. % to 50 wt. %, or 5 wt. % to 45 wt. %).
   Embodiment 50. The system of embodiment 49, wherein the metal oxide is an oxide of copper, chromium, molybdenum, vanadium, cerium, yttrium, scandium, tungsten, manganese, iron, cobalt, nickel, silver, bismuth, or any mixture thereof.
   Embodiment 51. The system of embodiment 49, wherein the metal oxide is copper oxide, or a mixture of copper oxide and one or more of manganese oxide and cerium oxide.
   Embodiment 52. The system of embodiment 49, wherein the particulate non-catalytic additive comprises
copper oxide, present in an amount within the range of 3 wt. % to 20 wt. %;
optionally, manganese oxide, present in an amount of up to 5 wt. %; and
optionally, cerium oxide, present in an amount of up to 10 wt. %.
   Embodiment 53. The system of any of embodiments 49-52, wherein the carrier is present in the particulate non-catalytic additive in an amount within the range of 40 wt. % to 99 wt. %.
   Embodiment 54. The system of any of embodiments 49-53, wherein the carrier comprises one or more of silica, alumina, calcium aluminate, and kaolin, present in a combined amount of at least 80 wt. % (e.g., at least 85 wt. %, at least 90 wt. %, at least 95 wt. %, at least 97.5 wt. %, or at least 99 wt. %) of the carrier.
   Embodiment 55. The system of any of embodiments 49-53, wherein the carrier comprises alumina, calcium aluminate, or a mixture thereof, present in a combined amount of least 80 wt. % (e.g., at least 85 wt. %, at least 90 wt. %, at least 95 wt. %, at least 97.5 wt. %, or at least 99 wt. %) of the carrier.
   Embodiment 56. The system of any of embodiments 49-55, wherein
the carrier (e.g., including alumina and/or calcium aluminate) is present in the non-catalytic additive in an amount within the range of 55 wt. % to 95 wt. %; and
the metal oxide (e.g., copper oxide, manganese, and/or cerium) is present in the non-catalytic additive in an amount within the range of 5 wt. % to 45 wt. %.
   Embodiment 57. The system of any of embodiments 49-56, wherein the heat-generating material and carrier comprise at least 70 wt. % (e.g., at least 80 wt. %, or at least 90 wt. %, or at least 95 wt. %) of the particulate non-catalytic additive.
   Embodiment 58. The system of any of embodiments 49-57, wherein the average particle size of the particulate non-catalytic additive is within the range of 5 μm to 5 mm (e.g., 5 μm to 1 mm, or 3 mm to 5 mm).
   Embodiment 59. The system of any of embodiments 49-58, wherein the particulate non-catalytic additive is present in the mixed-bed system in an amount within the range of 0.5 wt. % to 35 wt. % (e.g., 1 wt. % to 25 wt. %).
   Embodiment 60. The system of any of embodiments 1-59 contained in a reactor bed, wherein at least a portion (e.g., all) of the particulate non-catalytic additive comprises a layer over the particulate dehydrogenation catalyst such that a hydrocarbon feed introduced into the reactor first contacts the layer comprising particulate non-catalytic additive.
   Embodiment 61. The system of any of embodiments 1-60, wherein the ratio of particulate dehydrogenation catalyst and particulate non-catalytic additive present in the mixed-bed system is 99:1 to 3:2 (e.g., 19:1 to 1:1, or 9:1 to 2:3) by weight.
   Embodiment 62. The system of any of embodiments 1-61, wherein the particulate dehydrogenation catalyst and particulate non-catalytic additive are present in the mixed-bed system in a combined amount of at least 90 wt. % (e.g., at least 95 wt. %, or at least 98 wt. %, or at least 99 wt. %).
   Embodiment 63. A method for dehydrogenating hydrocarbons, the method comprising
providing the mixed-bed system of any of embodiments 1-62, the system comprising a oxidized heat-generating material; and
performing a plurality of reaction cycles, each reaction cycle comprising
   contacting a hydrocarbon feed with the system to dehydrogenate the hydrocarbon feed and to form a deactivated system comprising a reduced heat-generating material and reaction by-products (e.g., coke) adsorbed onto the surface of the mixed-bed system; and
   contacting the deactivated system with an oxygen-containing gas (e.g., air) to remove adsorbed reaction by-products (e.g., coke) and to oxidize the heat-generating material.
      Embodiment 64. The method of embodiment 63, wherein reduction of the heat-generating material provides heat to the system.
      Embodiment 65. The method of embodiment 63, wherein oxidation of the heat-generating material provides heat to the system.
      Embodiment 66. The method of embodiment 63, wherein
reduction of the heat-generating material provides heat to the system; and
oxidation of the heat-generating material provides heat the system.
   Embodiment 67. A method according to any of embodiments 63-66, wherein the hydrocarbon feed comprises one or more C₃-C₅ alkanes.
   Embodiment 68. A method according to any of embodiments 63-66, wherein the hydrocarbon feed comprises propane.
   Embodiment 69. A method according to any of embodiments 63-68, wherein the hydrocarbon feed is contacted with the catalyst at a space velocity within the range of 0.5 h⁻¹ to 4 h⁻¹ LHSV.
   Embodiment 70. A method according to any of embodiments 63-69, wherein the hydrocarbon feed is contacted with the catalyst is conducted at a temperature within the range of 400° C. to 750° C.
   Embodiment 71. A method according to any of embodiments 63-70, wherein the dehydrogenation is conducted at a pressure within the range of 0.1 bar to 1 bar.
   Embodiment 72. The method according to any of embodiments 63-71, wherein the temperature of the oxygen-containing gas is within the range of 400° C. to 750° C.

1. A mixed-bed system comprising
a particulate dehydrogenation catalyst comprising a primary species P1 selected from Ga, In, TI, Ge, Sn, Pb, and any mixture thereof as an active metal, disposed on a support; and
a particulate non-catalytic additive comprising a heat-generating material and a carrier selected from inorganic oxides, clays, and any mixture thereof.

2. A mixed-bed system comprising
a particulate dehydrogenation catalyst comprising
a primary species P1 selected from Ga, In, TI, Ge, Sn, Pb, and any mixture thereof, present in the particulate dehydrogenation catalyst in an amount within the range of 0.05 wt. % to 20 wt. %, calculated as elemental metal on a calcined basis;
a primary species P2 selected from the lanthanides and any mixture thereof, present in the particulate dehydrogenation catalyst in an amount within the range of 0.05 wt. % to 10 wt. %, calculated as elemental metal on a calcined basis;
a promoter M1 selected from Ni, Pd, Pt, La, Ir, Zn, Fe, Rh, Ru, Mn, Co, W, and any mixture thereof, present in the particulate dehydrogenation catalyst in an amount within the range of 1 ppm to 500 ppm, calculated as elemental metal on a calcined basis;
a promoter M2 selected from Li, Na, K, Rb, Cs, Be, Mg, Ca, Sr, Ba, and any mixture thereof, present in the particulate dehydrogenation catalyst in an amount within the range of 0.05 wt. % to 3 wt. %, calculated as elemental metal on a calcined basis; and
a support S1 selected from silica, alumina, zirconia, titania, yttria and any mixture thereof, present in the particulate dehydrogenation catalyst in an amount within the range of 50 wt. % to 99 wt. %, calculated as oxide on a calcined basis; and
a particulate non-catalytic additive comprising
a heat-generating material; and
a carrier selected from inorganic oxides, clays, and any mixture thereof.

3. The system of claim 2, wherein
P1 is Ga; and
P2 is selected from La, Ce, Nd, and any mixture thereof.

4. The system of claim 2, wherein
P1 is present in the particulate dehydrogenation catalyst in an amount within the range of 0.1 wt. % to 10 wt. % (e.g., 0.5 wt. % to 10 wt. %, or 2.5 wt. % to 5 wt. %), calculated as elemental metal on a calcined basis; and
P2 is present in the particulate dehydrogenation catalyst in an amount within the range of 0.1 wt. % to 6 wt. % (e.g., 0.5 wt. % to 6 wt. %, or 1 wt. % to 4 wt. %), calculated as elemental metal on a calcined basis.

5. The system of claim 2, wherein
M1 is selected from Pd, Pt, Ir, La, and any mixture thereof; and
M2 is selected from Li, Na, K, Cs, Ba, and any mixture thereof.

6. The system of claim 2, wherein
M1 is present in the particulate dehydrogenation catalyst in an amount within the range of 1 ppm to 500 ppm (e.g., 50 ppm to 400 ppm), calculated as elemental metal on a calcined weight basis; and
M2 is present in the particulate dehydrogenation catalyst in an amount within the range of 0.05 wt. % to 2.5 wt. % (e.g., 0.25 wt. % to 2.5 wt. %), calculated as elemental metal on a calcined basis.

7. The system of claim 2, wherein S1 includes a mixture of silica and alumina, present in a combined amount of at least 80 wt. % (e.g., at least 85 wt. %, at least 90 wt. %, at least 95 wt. %, at least 97.5 wt. %, or at least 99 wt. %) of S1.

8. The system of claim 2, wherein S1 is present in the particulate dehydrogenation catalyst in an amount within the range of 50 wt. % to 99 wt. % (e.g., 70 wt. % to 99 wt. %, or 90 wt. % to 99 wt. %).

9. The system of claim 2, wherein S1 comprises the calcined (e.g., at a temperature within the range of 500° C. to 1200° C.) product of a hydrolysis-poycondensation of one or more oxy compounds of silicon, aluminum, titanium, and/or zirconium (e.g., alkoxides, oxynitrates, and hydroxides).

10. The system of claim 2, wherein the total amount of P1, P2, M1, M2, and S1 is at least 80 wt. % (e.g., at least 85 wt. %, at least 90 wt. %, at least 95 wt. %, at least 97 wt. %, or at least 98 wt. %, or at least 99 wt. %, or at least 99.5 wt. %) of the particulate dehydrogenation catalyst.

11. The system of claim 2, wherein the particulate dehydrogenation catalyst comprises the calcined (e.g., at a temperature within the range of 500° C. to 1100° C.) product of impregnation of S1 with an impregnation solution (e.g., an aqueous impregnation solution) containing one or more of a P1 source, a P2 source, an M1 source, and an M2 source.

12. The system of claim 2, wherein the particulate non-catalytic additive comprises a metal oxide heat-generating material, present in an amount within the range of 0.5 wt. % to 60 wt. % (e.g., 1 wt. % to 50 wt. %, or 5 wt. % to 45 wt. %).

13. The system of claim 2, wherein the heat-generating material and carrier comprise at least 70 wt. % (e.g., at least 80 wt. %, or at least 90 wt. %, or at least 95 wt. %) of the particulate non-catalytic additive.

14. The system of claim 2, wherein
the particulate dehydrogenation catalyst is present in the mixed-bed system in an amount within the range of 65 wt. % to 99.5 wt. % (e.g., 75 wt. % to 99 wt. %); and
the particulate non-catalytic additive is present in the mixed-bed system in an amount within the range of 0.5 wt. % to 35 wt. % (e.g., 1 wt. % to 25 wt. %).

15. A method for dehydrogenating hydrocarbons, the method comprising
providing the mixed-bed system of claim 2, the system comprising a oxidized heat-generating material; and
performing a plurality of reaction cycles, each reaction cycle comprising
contacting a hydrocarbon feed with the system to dehydrogenate the hydrocarbon feed and to form a deactivated system comprising a reduced heat-generating material and reaction by-products (e.g., coke) adsorbed onto the surface of the mixed-bed system; and
contacting the deactivated system with an oxygen-containing gas (e.g., air) to remove adsorbed reaction by-products (e.g., coke) and to oxidize the heat-generating material.
